(19) United States
(12) Patent Application Publication
(10) Pub. No.: US 2006/0110478 A1
(43) Pub. Date: May 25, 2006

(54) Delivery system and method for supporting and promoting healthy sexual function and prevention and treatment of sexual dysfunction

(76) Inventors: Edward Larry McCleary, Incline Village, NV (US); Carl A. Forest, Boulder, CO (US); Christine McCleary, Incline Village, NV (US)

Correspondence Address: PATTON BOGGS, 1660 LINCOLN ST, SUITE 2050, DENVER, CO, 80264, US

(21) Appl. No.: 11/049,244

(22) Filed: Feb. 2, 2005

Related U.S. Application Data
(60) Provisional application No. 60/630,529, filed on Nov. 22, 2004.

(51) Int. Cl. A61K 36/489

(52) U.S. Cl. 424/757

(57) ABSTRACT

Improved delivery systems and delivery methods for supporting and promoting healthy sexual function, for preventing sexual dysfunction, or for treatment of sexual dysfunction. A compositions including one or more cGMP-specific PDE5 inhibitors and/or dopaminergic agonists is administered in the form of a breath-care strip, mint or lozenge, or a food or beverage product. The cGMP-specific PDE5 inhibitor comprises an ingredient selected from the group consisting of sophoflavescenol, vardenafil, tadalafil, and sildenafil. The dopaminergic agonist comprises apomorphine. Vitex agnus-castus extract, and one or more of lipoic acid, L-Arginine, folic acid, trimethylglycine, policosanol, carnitine, biotin, and acetyl L-Carnitine may also be included in the delivery vehicle.

### RELATED APPLICATIONS

This application claims the benefit of U.S. Provisional Patent Application No. 60/630529 filed Nov. 22, 2004, which is hereby incorporated by reference to the same extent as though fully disclosed herein.

### BACKGROUND OF THE INVENTION

1. Field of the Invention

This invention relates in general to compositions for supporting and promoting healthy sexual function, preventing sexual dysfunction, and treatment of sexual dysfunction, and in particular to a more effective delivery system for such compositions.

2. Statement of the Problem

Erectile dysfunction (ED) is a common disorder affecting about 30 million men in the U.S. alone. ED has been defined as the inability to achieve or maintain an erection sufficient for intercourse. The predominant etiology for this condition is vascular insufficiency. Studies have shown that during sexual arousal, nitric oxide molecules are released into the surrounding tissue from nerve endings and endothelial cells in the genitals. These nitric oxide molecules then cause the enzyme guanylate cyclase to produce cyclic guanosine monophosphate (cGMP) which lowers the level of intracellular calcium in the surrounding tissue and allows for the relaxation of vascular smooth muscle cells. In the penis, relaxation of the corpus cavernosal vascular smooth muscle cells permits increased blood flow into the cavernosal spaces. This expands the sinusoidal tissues in the corpora cavernosa. This expansion then restricts venous outflow. The combination of increased arterial inflow and diminished venous outflow leads to enhanced intra-cavernosal pressure thereby producing penile rigidity.

Studies investigating sexual dysfunction in couples reveal that up to 76% of women have complaints of sexual dysfunction. The sexual arousal response consists of vasocongestion in the pelvis, vaginal lubrication and expansion and swelling of the external genitalia. The primary features of female sexual dysfunction are lack of engorgement/swelling, lack of lubrication and lack of pleasurable genital sensation. Their consequences are reduced sexual desire, pain during intercourse and difficulty achieving orgasm. Since physiology analogous to the male sexual response is responsible for the female sexual functions, it is believed that there is a vascular basis for a significant proportion of patients with such symptoms.

Pharmaceutical products, primarily prescription drugs, for treatment of sexual dysfunction, particularly ED, are known. Many of these are based on agents that indirectly improve cGMP levels in genital tissue. The cellular concentration of cGMP is determined by the balance between its synthesis by guanylyl cyclase, and its breakdown to 5′-GMP by cyclic nucleotide phosphodiesterases (PDEs). Pharmacologic intervention designed to inhibit the enzyme cGMP-binding cGMP-specific phosphodiesterase type 5 (PDE5) to promote elevation of local cGMP concentration in genital tissue has proved to be a valuable strategy in ED. This is shown by the clinical efficacy of sildenafil citrate (Viagra: Pfizer Inc, New York, N.Y.), the prototype for the class of PDE5 inhibitors designed to elevate cGMP in the corpus cavernosum and thus produce an improvement in ED. These drugs include vardenafil, tadalafil, sildenafil and other agents which inhibit cyclic guanosine monophosphate (cGMP)-specific phosphodiesterase type 5 (PDE5). Apomorphine hydrochloride, and its variations, conventionally all referred to in the art as apomorphine, which is planned to be sold by Abbot Laboratories under the trademarks Uprima or Ixense, is a pharmaceutical that enhances sexual function by affecting the brain. Apomorphine is a dopaminergic agonist that, once absorbed and transported into the brain, initiates a chain of reactions that result in increased blood flow to the male genital organs and an erection.

It has been reported that rat cGMP-specific PDE5 is inhibited by a naturally occurring substance, sophoflavescenol, a C-8 prenylated flavonol. (See, H. J. Shin, H. J. Kim, J. H. Kwak, H. O. Chun, J. H. Kim, H. Park, D. H. Kim, and Y. S. Lee; A Prenylated Flavonol, Sophoflavescenol: A Potent and Selective Inhibitor of cGMP Phosphodiesterase 5. Bioorg Med Chem Lett. 2002 Sep. 2;12(17):2313-6. This article reports that the inhibitory activities of five flavonoids, kushenol H (1), kushenol K (2), kurarinol (3), sophoflavescenol (4) and kuraridine (5), isolated from Sophora flavescens (also S. flavescens) were measured against rat cGMP-specific PDE5 to identify cGMP PDE5 inhibitory constituents. However, sophoflavescenol, or any other related substances, have never been reported to be useful in humans.

While great progress has been made in compositions for supporting and promoting healthy sexual function, preventing sexual dysfunction, and treatment of sexual dysfunction, none of these compositions have been found to be broadly effective, and, in particular, have not been effective for women. It would be highly useful therefore, if a way could be found to increase the effectiveness of these and other such compositions.

### SUMMARY OF THE INVENTION

The invention solves the above and other problems by recognizing that sexual health and sexual dysfunction are complex conditions, and that good, consistent sexual response, particularly in females, not only requires appropriate physiological conditions, but also requires appropriate psychological environments. For example, a single inappropriate word or gesture can ruin a sexual experience for many individuals, particularly women. In one aspect, the invention overcomes the above and other problems by providing a method and apparatus for delivery of a sexual health or sexual dysfunction composition that is more appropriate to the sexual experience. Preferably, the invention provides a sexual health composition delivery vehicle and method that does not interfere with the psychological aspects of sexual activity, and preferably enhances the psychological aspects of sexual activity. In this aspect, the delivery vehicle comprises an edible film, a breath-care strip, mint or lozenge, or a food, beverage, spice, condiment or salad dressing or other functional food.

The invention also solves the above and other problems by providing nutraceutical-based sexual health compositions and methods of administrating them. As discussed in more detail below, nutraceuticals are compositions that promote health in humans and other animals, which contain ingredients that occur naturally and/or ingredients that are extracted from naturally-occurring substances. Since they are naturally occurring compounds from plants and animals that have evolved together with human beings, nutraceuticals are more likely to have fewer and less serious adverse side effects than synthetic pharmaceuticals to which humans have never been exposed.

In another aspect, the invention also solves the above problem by providing a sexual health composition that nurtures and/or sustains multiple metabolic pathways that contribute to sexual health, and for modulating (i.e., altering or normalizing) metabolic pathways which inhibit sexual health and lead to sexual dysfunction. In particular, as discussed briefly above and discussed in more detail below, there are at least five sets of metabolic pathways that affect sexual function: the metabolic pathways associated with the production of nitric oxide by the nerve endings and endothelial cells in the genitals; the pathways associated with the break down of nitric oxide before it can stimulate guanylate cyclase; the metabolic pathways associated with production of cyclic guanosine monophosphate (cGMP) by the enzyme guanylate cyclase; the pathways that cause breakdown of cGMP, and the pathways by which a dopaminergic agonist can enhance sexual health via the brain. The compositions according to the invention address one or more of these metabolic pathways, and preferably a plurality of these pathways.

The nutraceutical formulation is comprised of an agent, or a combination of agents, capable of enhancing cGMP in the genitalia of one in need of the therapy. The formulation is preferably admixed with a pharmaceutically acceptable carrier, diluent, or excipient. In another aspect, the present invention relates to a method of treating an individual with sexual dysfunction; the method comprising delivering a nutraceutical formulation that is capable of enhancing cGMP in the genitalia, and the formulation is preferably mixed with a pharmaceutically acceptable carrier, diluent or excipient. In some embodiments, the nutraceutical is combined with pharmaceutical sexual function or performance ingredients to improve the efficacy of the pharmaceutical.

The invention provides a composition for supporting and promoting healthy sexual function, for preventing human sexual dysfunction, or for treatment of human sexual dysfunction, the composition comprising an ingredient including a portion of or a constituent of the plant Sophora flavescens or an extract of the plant Sophora flavescens in an effective amount for supporting and promoting healthy sexual function, for preventing human sexual dysfunction, or for treatment of human sexual dysfunction. Preferably, ingredient comprises sophoflavescenol. Preferably, the ingredient comprises a portion of the root of the plant. Preferably, the ingredient comprises an extract of the plant. Preferably, the ingredient comprises an extract of the root of the plant. Preferably, the composition further comprises Vitex agnus-castus extract in an effective amount for supporting and promoting healthy sexual function, for preventing human sexual dysfunction, or for treatment of human sexual dysfunction. Preferably, the composition further comprises an ingredient that enhances the amount, or activity, of guanylate cyclase, an ingredient that enhances the production of nitric oxide, or an ingredient that inhibits the breakdown of nitric oxide; the ingredients included in effective amounts for supporting and promoting healthy sexual function, for preventing human sexual dysfunction, or for treatment of human sexual dysfunction. Preferably, the ingredient that enhances the amount, or activity, of guanylate cyclase comprises biotin, the ingredient that enhances the production of nitric oxide comprises a compound selected from the group consisting of L-arginine and policosanol, and the ingredient that inhibits the breakdown of nitric oxide comprises an anti-oxidant selected from the group consisting of idebenone, co-enzyme Q10, vitamin E, ALA (Alpha Lipoic Acid), vitamin C, carnosine, tocotrienols, flavonoids, ALC, vinpocetine, selenium, lycopene, creatine, arginine, taurine, cysteine, NADH, resveratrol, ginkgo biloba, oligomeric proanthocyanidins, and phenolic antioxidants and folic acid, folate, folinic acid and tetrahydrofolate. Preferably, the composition is contained in a vehicle selected from the group consisting of: nutritional supplements, edible films, breath-care strips, mints or lozenges, foods, and beverages.

In another aspect, the invention provides a method for supporting and promoting healthy sexual function, for preventing human sexual dysfunction, or for treatment of human sexual dysfunction, the method comprising orally, sublingually, transdermally, subcutaneously or parenterally administering to the human, for an effective period, a composition comprising an ingredient comprising a portion of the plant Sophora flavescens or an extract of the plant Sophora flavescens in an effective amount for supporting and promoting healthy sexual function, for preventing human sexual dysfunction, or for treatment of human sexual dysfunction. Preferably, the administering comprises delivering the composition in a vehicle selected from the group consisting of: nutritional supplements, edible films, breath-care strips, mints or lozenges, mouth sprays, dietary supplements, foods, and beverages.

In still another aspect, the invention provides a composition for supporting and promoting healthy sexual function, for preventing human sexual dysfunction, or for treatment of human sexual dysfunction, the composition comprising Vitex agnus-castus extract in an effective amount for supporting and promoting healthy sexual function, for preventing human sexual dysfunction, or for treatment of human sexual dysfunction. Preferably, the composition further comprises an ingredient that enhances the amount, or activity, of guanylate cyclase, an ingredient that enhances the production of nitric oxide, or an ingredient that inhibits the breakdown of nitric oxide; the ingredients included in effective amounts for supporting and promoting healthy sexual function, for preventing human sexual dysfunction, or for treatment of human sexual dysfunction. Preferably, the composition is contained in a vehicle selected from the group consisting of: edible films, breath-care strips or lozenges, foods, and beverages.

In yet another aspect, the invention provides a composition for supporting and promoting healthy sexual function, for preventing human sexual dysfunction, or for treatment of human sexual dysfunction, the composition comprising: a cGMP-specific PDE5 inhibitor ingredient; and Vitex agnus-castus extract ingredient; the ingredients included in the composition in effective amounts for supporting and promoting healthy sexual function, for preventing human sexual dysfunction, or for treatment of human sexual dysfunction. Preferably, the cGMP-specific PDE5 inhibitor ingredient comprises an ingredient selected from the group consisting of sophoflavescenol, vardenafil, tadalafil, and sildenafil. Preferably, the composition further comprises an ingredient that enhances the amount, or activity, of guanylate cyclase, an ingredient that enhances the production of nitric oxide, or an ingredient that inhibits the breakdown of nitric oxide; the ingredients included in effective amounts for supporting and promoting healthy sexual function, for preventing human sexual dysfunction, or for treatment of human sexual dysfunction. Preferably, the composition is contained in a vehicle selected from the group consisting of: nutritional supplements, edible films, breath-care strips or lozenges, foods, and beverages.

In still another aspect, the invention provides a method for supporting and promoting healthy sexual function, for preventing human sexual dysfunction, or for treatment of human sexual dysfunction, the method comprising orally or parenterally administering to the human, for an effective period, a composition comprising: a cGMP-specific PDE5 inhibitor ingredient; and a Vitex agnus-castus extract ingredient; the method including providing the ingredients in effective amounts for supporting and promoting healthy sexual function, for preventing human sexual dysfunction, or for treatment of human sexual dysfunction. Preferably, the cGMP-specific PDE5 inhibitor ingredient comprises an ingredient selected from the group consisting of sophoflavescenol, vardenafil, tadalafil, and sildenafil. Preferably, the administering comprises delivering the composition in a vehicle selected from the group consisting of: edible films, breath-care strips or lozenges, foods, and beverages.

In a different aspect, the invention also provides a product for supporting and promoting healthy sexual function, for preventing human sexual dysfunction, for preventing human sexual dysfunction, or for treatment of human sexual dysfunction, the product comprising: a delivery vehicle selected from the group consisting of edible films, breath-care strips, mints, and breath-care lozenges; and a sexual health or sexual dysfunction composition carried by the vehicle in an effective amount for supporting and promoting healthy sexual function, for preventing human sexual dysfunction, or for treatment of human sexual dysfunction. Preferably, the sexual health or sexual dysfunction composition comprises an ingredient selected from the group consisting of: a portion of the plant Sophora flavescens, an extract of the plant Sophora flavescens such as sophoflavescenol, or a Vitex agnus-castus extract, the ingredient provided in an effective amount for supporting and promoting healthy sexual function, for preventing human sexual dysfunction, or for treatment of human sexual dysfunction. Preferably, the composition further comprises an ingredient that enhances the amount, or activity, of guanylate cyclase, an ingredient that enhances the production of nitric oxide, or an ingredient that inhibits the breakdown of nitric oxide; the ingredients included in effective amounts for supporting and promoting healthy sexual function, for preventing human sexual dysfunction, or for treatment of human sexual dysfunction. Preferably, the ingredient that enhances the amount, or activity, of guanylate cyclase comprises biotin, the ingredient that enhances the production of nitric oxide comprises a compound selected from the group consisting of L-arginine and policosanol, and the ingredient that inhibits the breakdown of nitric oxide comprises an anti-oxidant selected from the group consisting of idebenone, co-enzyme Q10, vitamin E, ALA, vitamin C, carnosine, tocotrienols, flavonoids, ALC, vinpocetine, selenium, lycopene, creatine, arginine, taurine, cysteine, NADH, resveratrol, ginkgo biloba, oligomeric proanthocyanidins, and phenolic antioxidants and folic acid, folate, folinic acid and tetrahydrofolate. Preferably, the sexual health or sexual dysfunction composition comprises a cGMP-specific PDE5 inhibitor, such as vardenafil, tadalafil, and sildenafil. Preferably, the sexual health or sexual dysfunction composition comprises a dopaminergic agonist, such as apomorphine. Preferably, the edible film comprises pullulan.

The invention also provides a food or beverage product for supporting and promoting healthy sexual function, for preventing human sexual dysfunction, for preventing human sexual dysfunction, or for treatment of human sexual dysfunction, the product comprising: a food or beverage base; and a sexual health or sexual dysfunction composition in an effective amount for supporting and promoting healthy sexual function, for preventing human sexual dysfunction, or for treatment of human sexual dysfunction. Preferably, the composition selected from the group consisting of: a portion of the plant Sophora flavescens, an extract of the plant Sophora flavescens, sophoflavescenol, vardenafil, tadalafil, sildenafil, apomorphine, and Vitex agnus-castus extract. Preferably, the base is a beverage base and the beverage is selected from the group consisting of: soft drinks, tea, coffee, milk, fruit juices, and sports drinks. Preferably, the base is a beverage base and the beverage comprises an alcoholic beverage. Preferably, the base is a food base and the food is selected from the group consisting of: fruit, chocolate, candy, trail mix, nutritional bars, ice cream, yogurt, cheese, steak sauce, salsa and salad dressing.

The invention further provides a method of administering a composition for supporting and promoting healthy sexual function, for preventing human sexual dysfunction, for preventing human sexual dysfunction, or for treatment of human sexual dysfunction, the method comprising: incorporating the composition in an edible film, breath-care strip, mint, or breath-care lozenge; and placing the edible film, breath-care strip, mint, or breath-care lozenge in the mouth of a human being.

The invention not only provides a nutraceutical-based sexual health composition and method of delivering it that has fewer adverse reactions than pharmaceutical-based sexual health compounds, but one that is more broadly effective, more potent than prior art pharmaceutical sexual health compounds, and more psychologically appropriate. Numerous other advantages and features of the invention will become apparent from the following detailed description.

### BRIEF DESCRIPTION OF THE DRAWING

FIG. 1 is a block diagram illustrating the food or beverage product according to the invention and the method of making it.

### DETAILED DESCRIPTION OF THE PREFERRED EMBODIMENT

1. Overview

The invention relates to certain compositions that promote healthy sexual function and/or sexual well being in humans and other animals and which contain ingredients that occur naturally and/or ingredients that are extracted from naturally occurring substances. We shall label these compositions as “nutraceuticals” herein. Depending on the point of view of the persons using or prescribing them, nutraceuticals may be called: functional foods, dietary supplements, nutritional supplements, herbal supplements, botanical drugs, medical foods, and sometimes even drugs. In the future, other terms may be applied to these compounds, if, for example, Congress creates new categories of FDA-regulated products. The invention is intended to cover the compositions disclosed herein, no matter what they are called. Nutraceuticals do not include pharmaceuticals, which are defined herein to be man-made, synthetic medical compounds that do not occur in nature.

As stated above, the present invention provides a method and composition for maintaining sexual health, for preventing sexual dysfunction, and for treatment of sexual dysfunction in humans or other animals, such as horses. The composition is preferably a nutraceutical or a combination of nutraceuticals, although in one aspect it could includes one or more pharmaceuticals. In the most preferred embodiments, it includes a combination of ingredients that address a plurality of metabolic pathways, and thus is more broadly effective than prior art sexual function compositions.

As mentioned above, the cellular concentration of cGMP is determined by the balance between its synthesis by guanylyl cyclase, and its breakdown to 5′-GMP by cyclic nucleotide phosphodiesterases (PDE5). The synthesis is stimulated by nitric oxide molecules that are released into the surrounding tissue from nerve endings and endothelial cells in the genitals. Without being exhaustive, the enhanced production of cGMP may be achieved by enhancement of the amount, or activity, of guanylate cyclase, the enhancement of the production of nitric oxide, the inhibition of the breakdown of nitric oxide, and the inhibition of the breakdown of cGMP. The compositions according to the invention address one or more of these metabolic pathways, and preferably a plurality of these pathways. They also address the impact of brain pathways, such as the dopaminergic agonist pathway, upon sexual function.

The inhibition of the breakdown of cGMP is achieved either by sophoflavescenol or other extract from the plant Sophora flavescens, or other selective inhibitor of cyclic guanosine monophosphate (cGMP)-specific phosphodiesterase type 5 (PDE5). Preferably, the PDE5 inhibitor is an extract from Sophora flavescens, more preferably an extract from the root of Sophora flavescens, and most preferably, sophoflavescenol. Sophoflavescenol is a selective cGMP PDE5 inhibitor that is not structurally related to the pharmaceutical cGMP PDE5 inhibitors that have been synthesized by modification of the sildenafil structure. Therefore, sophoflavescenol may be considered as a unique agent with a different, more desirable pharmacological PDE5 profile.

As discussed above, sophoflavescenol, a C-8 prenylated flavonol, is isolated by methanol extraction of the roots of Sophora flavescens. The methanol extract of S. flavescens is suspended in water and then partitioned consecutively with dichloromethane, ethyl acetate, and butanol. The ethyl acetate fraction contains the most potent inhibitory activity against cGMP PDE5 (IC50=1.54 μg/ml). Of the flavonols in the ethyl acetate fraction, sophoflavescenol is the most potent. In addition, the IC50 ratio PDE4/PDE5 is 196.2 showing high selectivity in the inhibition of these PDE isoforms.

The structure of sophoflavescenol is shown below: 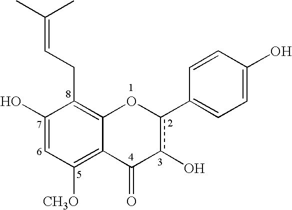
It is not structurally related to the other cGMP PDE5 inhibitors, such as the vardenafil salts. The present document discloses for the first time compositions and methods whereby sophoflavescenol may be used directly and alone, or may serve as the lead ingredient for the formulation of compositions effective for sexual function and performance. Since it is a naturally-occurring compound from the root of a plant that has grown wild for centuries and has been cultivated for generations, it is likely to have more desirable pharmacological profiles than pharmaceuticals to which humans have never been exposed. As will be discussed in detail below, it is preferably combined with other synergistically acting agents for enhanced function.

Sophoflavescenol elevates cGMP in genital tissue by inhibiting cGMP breakdown. It may be used alone, or in combination with other biologically active compounds that directly, or indirectly, increase the production of cGMP and/or inhibit its breakdown, and thus act synergistically to enhance sexual function. Without being exhaustive, the enhanced production of cGMP may be achieved by augmentation of the amount, or activity, of guanylate cyclase, the augmentation of the production of nitric oxide, the inhibition of the breakdown of nitric oxide or the providing of a dopaminergic agonist.

The increased production of nitric oxide may be achieved by increased supply of metabolic substrate of the enzyme Nitric Oxide Synthase (NOS), and by provision of agents that augment the activity or amount of NOS. Nitric oxide readily combines with the superoxide radical to form peroxynitrate. This and other related reactions consume nitric oxide. Inactivation of nitric oxide may be diminished by the use of anti-oxidants, such as idebenone, co-enzyme Q10, vitamin E, ALA, vitamin C, carnosine, tocotrienols, flavonoids, ALC, vinpocetine, selenium, lycopene, creatine, certain amino acids (e.g., taurine and cysteine), resveratrol, ginkgo biloba, oligomeric proanthocyanidins, and phenolic antioxidants, and related compounds such as folic acid, folate, folinic acid and methyltetrahydrofolate. These agents may be used singly, or combined, by one skilled in the art, such as a physician, to produce maximal cGMP levels in the genital tissues with subsequent beneficial activity for sexual function and performance and to address sexual dysfunction.

As discussed above, the physiologic key to addressing many of the factors involved in the development of sexual function is vascular health and vascular function. These factors are key mediators in the control of blood flow to the genitalia. The vascular abnormality that is most commonly associated with sexual dysfunction relates to a diminished ability to accumulate cGMP via the nitric oxide synthesis pathway. It involves the endothelium, the cells that form the inner lining of all blood vessels. This is referred to as endothelial dysfunction.

During the aging process, associated with vascular disease, or related to a number of medical conditions such as diabetes, menopause or hyper-homocyst(e)inemia, adequate nitric oxide may not be produced, or it may be broken down into compounds that actually damage blood vessels further contributing to vascular disease and endothelial dysfunction. Superoxide overproduction is a prominent mediator of endothelial dysfunction acting in a number of ways to inhibit effective endothelial nitric oxide activity. The ability of superoxide to quench nitric oxide is well known, but oxidants derived from superoxide also appear to inhibit dimethylarginine dimethylaminohydrolase (DDAH) and to oxidize tetrahydrobiopterin (THBP). The former action boosts the level of methylated arginines that act as potent competitive inhibitors of NOS, whereas the latter effect decreases the ability of the enzyme to generate nitric oxide, while converting it to a form that readily generates further superoxide. The adverse impact of DDAH deficiency on nitric oxide production may be offset with supplemental arginine. A practical way to optimize NOS activity in the context of THBP-depleted endothelial cells is the administration of high dose folic acid independent of modulation of homocyst(e)ine levels. The administration of either arginine or folate alone, or preferably simultaneously, is effective in preventing or ameliorating vascular pathology that contributes to sexual dysfunction.

Excessive endothelial superoxide production can be addressed by controlling risk factors that boost activation of endothelial NAD(P)H oxidase such as insulin resistance, diabetes, hypercholesterolemia, and hypertension. Supplemental administration of lipoic acid and other anti-oxidants mentioned above, anti-reductants, and policosanol are helpful in this regard.

The brain's function as a sexual enhancer is preferably addressed by Vitex agnus-castus. The inventors have found that Vitex agnus-castus appears to be a dopaminergic agonist. The function of Vitex agnus-castus is not yet fully understood, and may be more complex than just as a dopaminergic agonist. The invention contemplates that, whatever sexual health and performance enhancement function or functions associated with Vitex agnus-castus are eventually determined to be, these sexual health and performance enhancement functions are considered to be part of this invention.

2. The Preferred Nutraceutical Sexual Health Compositions

Using the above general guidelines, compositions effective in supporting and promoting healthy sexual function and in addressing sexual dysfunction have been formulated. Table 1 lists the preferred ingredients of the compositions, the dosage range, and the preferred dose of each ingredient.

TABLE I

| Agent | Dose | Preferred Dose |
|---|---|---|
| Sophoflavescenol | 10 μg to 5 g | 1 mg to 1 g |
| Lipoic Acid | 10 mg to 1 g | 25 mg to 600 mg |
| Vitamin C | 10 mg to 5 g | 25 mg to 3 g |
| Vitamin E | 1 IU-2000 IU | 10 IU-1000 IU |
| Trimethylglycine | 10 mg to 2 g | 50 mg to 1 g |
| Folic Acid, Folate, Folinic Acid, | 200 μg to 10 mg | 400 μg to 5 mg |
| Methyl Tetrahydrofolate | | |
| Carnitine | 10 mg to 1 g | 25 mg to 500 mg |
| Acetyl L-Carnitine | 10 mg to 1 g | 25 mg to 500 mg |
| L-Arginine | 10 mg to 20 g | 50 mg to 10 g |
| Policosanol | 1 mg to 30 mg | 3 mg to 15 mg |
| Biotin | 10 mcg to 10 mg | 200 mcg to 5 mg |
| Vitex agnus-castus extract | 1 μg to 10 g | 4 μg to 5 g |
| (standardized to casticin) | | |
| Kushenol H | 1 μg to 100 g | 1 mg to 50 g |
| Kushenol K | 1 μg to 100 g | 1 mg to 50 g |
| Kuraridine | 1 μg to 100 g | 1 mg to 50 g |
| Kurarinol | 1 μg to 100 g | 1 mg to 50 g |

One skilled in the art can formulate the preferred composition for any particular individual and the preferred method of administration of the composition based upon his or her skill, awareness of the medical literature, needs of the subject, guidance given in this disclosure, and the desired effect. To achieve a successful result, general considerations that one skilled in the art should follow are:
1) Most formulations will preferably include an appropriate amount of sophoflavescenol or other Sophora flavescens extract;
2) Most formulations will include Vitex agnus-castus extract;
3) Depending upon the clinical circumstances and the insight of the formulator, appropriate amounts of the remaining agents are chosen. None of them, one of them, a combination of some of them, or all may be used.
4) Appropriate delivery constituents should be chosen;
5) Mode of delivery and delivery regimen should be specified—this may include oral, sublingual, transdermal, subcutaneous application, or intra-venous administration and may vary from an as-needed basis to taking a preferably lower dosage several times a day. In particular, the delivery vehicles described in section 3 below, should be considered.

Exemplary formulations are given below to illustrate the application of the principals of the invention to a variety of situations.

### EXAMPLES 1

Healthy Male with ED

Composition A: Sophoflavescenol 100 mg; given orally only when needed.
Composition B: Sophoflavescenol 75 mg combined with Vitex agnus-castus extract 30 mg; given orally as needed.

### EXAMPLE 2

Diabetic Male with ED

Composition C: Sophoflavescenol 100 mg
   Lipoic Acid 100 mg
   Trimethylglycine 500 mg
   Given orally on a daily basis.

### EXAMPLES 3

Aging Male with ED

Composition D: Sophoflavescenol 150 mg
   Alpha Lipoic Acid 125 mg
   L-Arginine 500 mg
   Folic Acid 400 μg
   Policosanol 5 mg
   Vitex agnus-castus extract 15 mg
   Given orally on a daily basis.
Alternative composition: Biotin 1.2 mg may also be added to each dose.

### EXAMPLE 4

Paraplegic Male

Composition E: Sophoflavescenol 200 mg
   Lipoic Acid 300 mg
   L-Arginine 500 mg
   Folic Acid 800 μg
   Trimethylglycine 500 mg
   Carnitine 500 mg
   Acetyl L-Carnitine 500 mg
   Given orally as needed.

### EXAMPLES 5

38 y/o Female with Sexual Dysfunction

Composition F: Sophoflavescenol 150 mg
   Given orally as needed
Composition G: To the above may also be added:
   Biotin 800 mcg
   And/or Vitex agnus-castus extract 20 mg

### EXAMPLE 6

Postmenopausal Female

Composition H: Sophoflavescenol 250 mg

Formulated in a gel base and applied directly to the genitalia as needed.

### EXAMPLES 7

The following formulations may be tried with persons having clear sexual dysfunction symptoms but for which PDE5 inhibitors have little effect and/or have significant side effects;
Composition I: Lipoic Acid 125 mg
   L-Arginine 500 mg
   Folic Acid 400 μg
   Policosanol 5 mg
   Vitex agnus-castus extract 15 mg
   Vitamin C 100 mg
   Vitamin E 100 IU
   Biotin 800 mcg
   Given orally on a daily basis.
Composition J: Lipoic Acid 125 mg
   L-Arginine 500 mg
   Folic Acid 400 μg
   Policosanol 5 mg
   Vitex agnus-castus extract 15 mg
   Vitamin C 100 mg
   Vitamin E 100 IU
   Biotin 800 mcg
   Trimethylglycine 250 mg
   Carnitine 500 mg
   Acetyl L-Carnitine 500 mg
   Given orally on a daily basis.

Each of the above formulations and methods of administration should be considered only to be the starting point for addressing the indicated condition. As known in the art, if the starting formulation is not effective or not as effective as desired, the formulation should be modified by increasing dosages and/or adding additional ingredients. A good rule to follow in altering the formulations above that do not include Vitex agnus-castus extract is that the alternative of adding Vitex agnus-castus extract to the formulation should be considered before increasing dosages. If there are side effects, the formulation should be modified by dropping ingredients and/or dosages. For example, any of the anti-oxidants given above may be substituted for the vitamins C and E or used to augment the vitamins C and E. If formulations given on a daily basis are effective but have side effects, then they may be tried on an as-needed basis.

A key ingredient in the above and below compositions is sophoflavescenol. Sophoflavescenol is extracted using a methanol extraction from the root (radix) of the plant Sophora flavescens. There are also other active ingredients in the plant that are not as potent or effective, nor as specific at inhibiting only one specific isoform of the enzyme (cGMP type Phosphodiesterase 5) that produces the desired physiologic response. The invention contemplates that any of these ingredients may be substituted or used in combination with sophoflavescenol. In particular, the root of the plant may be used. More preferred forms would include extracts of the plant, and most preferably the following specific ingredients: Kushenol H, Kushenol K, Kuraridine, Kurarinol. The lower limit for the root, any extract of the plant, and the forgoing specific ingredients is the same as those indicated for sophoflavescenol, while the upper limit may be increased up to 1000 fold over the dose of sophoflavescenol.

Vitex agnus-castus extract is a key ingredient of both the nutraceutical formulations and the formulations that include pharmaceutical drugs. It was initially only one of hundreds of possible ingredients considered by the inventors for inclusion in a healthy sexual function formulation. However, initial testing has suggested that this ingredient broadens and increases the effectiveness of almost any formulation. The reason for this is not yet fully understood, but it is believed to be connected to the fact that it addresses metabolic pathways other than the PDE5 pathway.

The above compositions each address different metabolic pathways associated with sexual health. If the pathway that needs to be modulated is known, then this should be used as a guide to determine which composition is selected, according to the general guidelines given in the Overview above. If the pathway that needs to be modulated is not known, then it is recommended that the compositions A, B, C, D, E, I, and J be tried in order before increased dosages be tried.

Any of the compositions described above may also be combined with an erectile dysfunction drug or agent, such as a monohydrochloride salt of vardenafil (mVardenafil HCl), sildenafil citrate, tadalafil, or other selective inhibitor of cyclic guanosine monophosphate (cGMP)-specific phosphodiesterase type 5 (PDE5). MVardenafil HCl is designated chemically as piperazine, 1-[[3-(1,4-dihydro-5-methyl-4-oxo-7-propylimidazo [5,1-f][1,2,4]triazin-2-yl)-4-ethoxyphenyl]sulfonyl]-4-ethyl-, monohydrochloride and is sold by Bayer Pharmaceuticals under the trademark Levitra. Sildenafil citrate is designated chemically as 1-[[3-(6,7-dihydro-1methyl-7-oxo-3-propyl-1H-pyrazolo[4,3-d]pyrimidin-5-yl)4ethoxyphenyl]sulfonyl]-4-methyl piperazine citrate and is sold by Pfizer, Inc. under the trademark Viagra. Tadalafil is sold under the trademark Cialis by Lilly ICOS LLC, and is designated chemically as pyrazino[1¢,2¢:1,6]pyrido[3,4-b]indole-1,4-dione, 6-(1,3-benzodioxol-5-yl)-2,3,6,7,12,12a-hexahydro-2-methyl-, (6R,12aR)-. Many variations of such drugs also have been found to be similarly effective. Herein, as is conventional in the art, we shall refer to all variations of mVardenafil HCl that are effective to inhibit PDE5 as vardenafil, all effective variations of sildenafil citrate as sildenafil, and all effective variations of the tadalafil, as tadalafil. In addition, vardenafil, sildenafil, tadalafil, or any erectile dysfunction drug that addresses the PDE5 pathway, may be substituted for sophoflavescenol in any of the above compositions. The initial dosages for the ED drug should be the smaller dosages directed by the manufacturer.

Apomorphine hydrochloride, and its variations, conventionally all referred to in the art as apomorphine, which is planned to be sold by Abbot Laboratories under the trademarks Uprima or Ixense, is a pharmaceutical that enhances sexual function by affecting the central nervous system. Apomorphine is a dopamine receptor agonist that acts on the central nervous system. Once absorbed and transported into the brain, it initiates a chain of reactions that result in increased blood flow to the male genital organs and an erection. Apomorphine can also be added to any of the formulations given above.

The compositions presented above are preferably in the form of an orally administered composition, e.g., powder, chewable wafer, tablet, regular or compressed capsule, etc., wherein the amounts listed may be divided into two or more portions which in combination constitute a single “unit dose” of the composition. Each dose is preferably taken with 8 ounces of water.

The method of this invention involves the steps of administering to a human on a daily basis for a therapeutically effective period of time an effective amount of the composition of this invention. The composition is administered orally, sublingually, transdermally, subcutaneously or parenterally, preferably orally and ingested.

As used herein with respect to the amount of the composition used in the method of this invention, the term “effective amount” means an amount sufficient to modulate metabolic pathways in the body so as to enhance sexual health. Preferably, the active-ingredient composition (not including inactive ingredients) of this invention is administered in a daily dosage of at least about 1 gram, more preferably from about 1 gram to about 200 grams, most preferably from about 4 grams to about 20 grams. When inactive ingredients are present in the composition, the inactive ingredients of the composition can be present in any conventional amount used in orally or parenterally administered compositions.

The term “effective period of time” with respect to the administration of the composition in the method of this invention means that period of time sufficient to modulate healthy sexual function and performance in the human. The “effective period” may be a single time prior to sexual activity. However, preferably, the nutraceutical compositions of this invention can administered on a daily basis for a period of at least three weeks, more preferably at least six weeks.

As stated above, oral administration is accomplished by ingesting the composition, preferably with water. The orally administered composition of this invention can be in any conventional form including, e.g., capsules (regular or compressed), tablets, soft gels, chewable wafers, elixirs, liquid extracts, powders, granules, suspensions in water or non-aqueous media, sachets, etc. Powder, tablet, and chewable wafer forms are most preferred. The composition can also be administered parenterally. The alternative delivery vehicles discussed below should also be considered.

3. The Psychologically Appropriate Delivery System

A. Overview:

The inventors have noticed that the manufactures and marketers of prior art sexual performance and sexual dysfunction compositions, particularly the manufacturers and marketers of pharmaceuticals, appear to have a non-realistic and romanticized view of sexual arousal and function. Commercials show older, but sexually vibrant women who appear to have abnormally strong and persistent sex drives. The manufacturers and marketers of pharmaceuticals appear to think that sexual encounters can be planned—you take your ED pill two to three hours before the planned encounter, and everything goes off just as planned.

Nothing could be further from the truth. In reality, older persons experienced in sex know that sexual encounters cannot be planned. Planning, in fact, seems adverse to sex. Nature, in its continuous drive to diversity, seems to have designed sex so that the factors that result in a successful sexual encounter come together almost randomly. Before the individual with an undesirable sexual condition realizes it, the opportunity is upon him or her. Popping a pill in such a spontaneous situation is a reminder of previous impotency, for both parties, and is as effective as a cold shower. To a sexual psychologist, the low effectiveness of a sexual dysfunction pill comes as no surprise.

The invention overcomes the above problem by providing a delivery system and method that is not adverse to the sexual experience, and preferably enhances the sexual experience.

In one embodiment, the delivery system comprises a breath-care element. The breath-care product can be an actual or simulated breath-care element. An actual breath care element is one that contains one or more ingredients designed to enhance or sweeten the breath. Such ingredients include, for example, a pH control agent, such as urea and buffers, a sulfur precipitating agent to reduce bad breath odor, inorganic components for tartar or caries control, such as phosphates and fluorides, a breath freshening agent, such as zinc gluconate, an anti-plaque/anti-gingivitis agent, such as chlorohexidene, CPC, and triclosan, a saliva stimulating agent including, for example, food acids such as citric, lactic, maleic, succinic, ascorbic, adipic, fumaric and tartaric acids, antimicrobially effective amounts of essential oils such as thymol, methyl salicylate, eucalyptol and menthol, or other like breath enhancement ingredients or combinations thereof. A simulated breath-care product is one that has an appearance of a breath-care product, but does not actually contain any ingredient to enhance the breath. An example of a simulated breath-care product is an edible strip, mint or lozenge without a breath-care ingredient. In the most preferred form of this embodiment, the delivery system and method comprises a water soluble edible film. Preferably, the film comprises pullulan. The film may or may not include breath enhancers. The invention also contemplates that the breath-care element can also be a lozenge, mint, candy or other breath-care element. This embodiment lends itself particularly to sexual performance and sexual dysfunction compositions that are intended for one-time use just prior to a sexual encounter, though it also can be used to deliver compositions that are regularly administered. Since the use of a breath care product is natural in social situations, this method of delivery does not create a psychological impediment to sex. In fact, it can enhance the possibility of sex, since use of a breath-care product reflects consideration for one's partner.

In another embodiment, the delivery system comprises a food, beverage, spice, condiment or salad dressing. The preferred delivery systems include sports beverages, energy bars, candy (such as chocolate), shakes, alcoholic beverages and salad dressings. This embodiment lends itself particularly to sexual health and sexual dysfunction compositions that are intended to be administered on a regular basis, such as daily. However, they also can be used for one-time compositions, since food and beverages blend well with sexual activity.

In confidential pre-design testing, some comments concerning the food, beverage, spice, condiment or salad dressing embodiment above added to the inventor's consideration that the prior art approach to delivery of sexual health and sexual dysfunction composition was divorced from reality. Some individuals commented that the regular, such as daily, administration of sexual dysfunction compositions seemed unnatural. In fact, however, this is the more natural approach. Nature has designed the human sexual function so that persons with normal sexual health are generally able to perform sexual acts at any time that sexual stimulation is present. For that reason, it is believed that this approach will be the most effective approach.

B. Description of the Preferred Edible Film Delivery Vehicle

The preferred breath-care element delivery system comprises an edible film. The preferred edible film comprises pulullan. Edible films are well-known in the art. For example, see: U.S. Pat. No. 6,596,298 B2 issued Jul. 22, 2003 to Leung et al; U.S. Pat. No. 6,656,493 B2 issued Dec. 2, 2003 to Dzija et al., U.S. Pat. No. 5,411,945 issued May 2, 1995 to Ozaki et al., particularly example B-2; U.S. Pat. No. 5,518,902 issued May 21, 1996 to Ozaki et al.; and International Patent Application Publication No. WO 99/17753; all of which are hereby incorporated by reference to the same extent as though fully disclosed herein.

The film-forming agent used in the films according to the present invention can be selected from the group consisting of pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof. Pullulan is used in amounts ranging from about 0.01 to about 99 wt %, preferably about 30 to about 80 wt %, more preferably from about 45 to about 70 wt % of the film and even more preferably from about 60 to about 65 wt % of the film.

Sulfur precipitating agents, pH control agents, tartar or caries control agents, breath freshening agents, anti-plaque/anti-gingivitis agents, antimicrobially agents, saliva stimulating agents, cooling agents, plasticizing agents, surfactants, stabilizing agents, emulsifying agents, thickening agents, and binding agents can also be added to the films.

Sulfur precipitating agents bind with, and inactivate, the volatile sulfur compounds that cause a large percentage of oral malodor. Sulfur precipitating agents useful in the present invention include metal salts such as copper salts and zinc salts. Preferred salts include copper gluconate, zinc citrate and zinc gluconate. The amount of sulfur precipitating agent is from about 0.01 to about 2 wt %, preferably about 0.15 wt % to about 1.5 wt %, even more preferably about 0.25 wt % to about 1.0 wt % of the film. PH control agents include urea and buffers. Tartar or caries control agents include phosphates and fluorides. Breath freshening agents include zinc gluconate, anti-plaque/anti-gingivitis agents include chlorohexidene, CPC, and triclosan. Antimicrobially agents include effective amounts of essential oils such as thymol, methyl salicylate, eucalyptol and menthol.

Saliva stimulating agents are disclosed in U.S. Pat. No. 4,820,506, which is incorporated by reference as though fully disclosed herein. These include food acids such as citric, lactic, malic, succinic, ascorbic, adipic, fumaric and tartaric acids, preferably, citric, malic and ascorbic acids. The amount of saliva stimulating agents in the film is from about 0.01 to about 12 wt %, preferably about 1 wt % to about 10 wt %, even more preferably about 2.5 wt % to about 6 wt %.

Cooling agents include monomenthyl succinate, preferably in amounts ranging from about 0.001 to about 2.0 wt %, preferably about 0.2 to about 0.4 wt %. A monomenthyl succinate containing cooling agent is available from Mane, Inc. Other suitable cooling agents include WS3, WS23, Ultracool II and the like. Plasticizing agents include triacetin, preferably in amounts ranging from about 0 to about 20 wt %, preferably about 0 to about 2 wt %. Other suitable plasticizing agents include monoacetin and diacetin.

Preferred surfactants include mono and diglycerides of fatty acids and polyoxyethylene sorbitol esters, such as, Atmos 300 and Polysorbate 80. The surfactant can be added in amounts ranging from about 0.5 to about 15 wt %, preferably about 1 to about 5 wt % of the film. Other suitable surfactants include pluronic acid, and sodium lauryl sulfate. Stabilizing agents include xanthan gum, locust bean gum, carrageenan, and guar gum, preferably in amounts ranging from about 0 to about 10 wt %, preferably about 0.1 to about 2 wt % of the film. Emulsifying agents include triethanolamine stearate, quaternary ammonium compounds, acacia, gelatin, lecithin, bentonite, veegum, and the like, preferably in amounts ranging from about 0 to about 5 wt %, preferably about 0.01 to about 0.7 wt % of the film. Thickening agents include methylcellulose, carboxyl methylcellulose, and the like, preferably in amounts ranging from about 0 to about 20 wt %, preferably about 0.01 to about 5 wt %. Binding agents include starch, preferably in amounts ranging from about 0 to about 10 wt %, preferably about 0.01 to about 2 wt % of the film.

It is well known in the art to add sweeteners to an edible film, particularly a breath-care film. Both natural and artificial sweeteners can be used. Suitable sweeteners include: water-soluble sweetening agents such as monosaccharides, disaccharides and polysaccharides such as xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin; water-soluble artificial sweeteners such as the soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), and the free acid form of saccharin; dipeptide based sweeteners, such as L-aspartic acid derived sweeteners, such as L-aspartyl-L-phenylalanine methyl ester (aspartame) and materials described in U.S. Pat. No. 3,492,131, L-alpha-aspartyl-N-(2,2,4,4-tetramethyl-3-thietanyl)-D-alaninamide hydrate, methyl esters of L-aspartyl-L-phenylglycerin and L-aspartyl-L-2,5, dihydrophenyl-glycine, L-aspartyl-2,5-dihydro-L-phenylalanine, and L-aspartyl-L-(1-cyclohexyen)-alanine; water-soluble sweeteners derived from naturally occurring water-soluble sweeteners, such as a chlorinated derivative of ordinary sugar (sucrose), known, for example, under the name sucralose; and protein based sweeteners such as thaumatoccous danielli (Thaumatin I and II). Another naturally sweet substance is the herb stevia, used as an ingredient in dietary supplements. In general, an effective amount of auxiliary sweetener is utilized to provide the level of sweetness desired for a particular composition, and this amount will vary with the sweetener selected. This amount will normally be 0.01% to about 10% by weight of the composition when using an easily extractable sweetener. The water-soluble sweeteners described above, are usually used in amounts of about 0.01 to about 10 wt %, and preferably in amounts of about 2 to about 5 wt %. Some of these sweeteners, e.g., glycyrrhizin, as well as the other sweeteners mentioned are generally used in amounts of about 0.01 to about 10 wt %, with about 2 to about 8 wt % being preferred and about 3 to about 6 wt % being most preferred. These amounts may be used to achieve a desired level of sweetness independent from the flavor level achieved from any optional flavor oils used.

Flavorings may also be added as known in the art. These flavorings may include synthetic flavor oils and flavoring aromatics, and/or oils, oleo resins and extracts derived from plants, leaves, flowers, fruits and so forth, and combinations thereof. Representative flavor oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, including lemon! orange, grape, lime and grapefruit and fruit essences including apple, pear, peach, strawberry, raspberry, cherry, plum, pineapple, apricot and so forth. These flavorings can be used individually or in admixture. Commonly used flavors include mints such as peppermint, artificial vanilla, cinnamon derivatives, and various fruit flavors, whether employed individually or in admixture. Flavorings such as aldehydes and esters including cinnamyl acetate, cinnamaldehyde, citral, diethylacetal, dihydrocarvyl acetate, eugenyl formate, p-methylanisole, and so forth may also be used. Generally, any flavoring or food additive, such as those described in Chemicals Used in Food Processing, publication 1274 by the National Academy of Sciences, pages 63-258, may be used. Further examples of aldehyde flavorings include, but are not limited to acetaldehyde (apple); benzaldehyde (cherry, almond); cinnamic aldehyde (cinnamon); citral, i.e., alpha citral (lemon, lime); neral, i.e. beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 2,6-dimethyl-5-heptenal, i.e. melonal (melon); 2-6-dimethyloctanal (green fruit); and 2-dodecenal (citrus, mandarin); cherry; grape; mixtures thereof; and the like. The amount of flavoring employed is normally a matter of preference subject to such factors as flavor type, individual flavor, and strength desired. Thus, the amount may be varied in order to obtain the result desired in the final product. Such variations are within the capabilities of those skilled in the art without the need for undue experimentation. In general, amounts of about 0.1 to about 30 wt % are useable with amounts of about 2 to about 25 wt % being preferred and amounts from about 8 to about 10 wt % are more preferred.

Coloring agents or colorants also may be used in amounts effective to produce the desired color. These include pigments such as titanium dioxide, which may be incorporated in amounts of up to about 5 wt %, and preferably less than about 1 wt %. Colorants can also include natural food colors and dyes suitable for food, drug and cosmetic applications. These colorants are known as FD&C (Food, Drug and Cosmetic Act) dyes and lakes. The materials acceptable for the foregoing spectrum of use are preferably water-soluble, and include FD&C Blue No. 2, which is the disodium salt of 5,5-indigotindisulfonic acid. Similarly, the dye known as Green No. 3 comprises a triphenylmethane dye and is the monosodium salt of 4-[4-N-ethyl-p-sulfobenzylamino)diphenyl-methylene]-[1-N-ethyl-N-p-sulfonium benzyl)-2,5-cyclo-hexadienimine]. A full recitation of all FD&C and D&C dyes and their corresponding chemical structures may be found in the Kirk-Othmer Encyclopedia of Chemical Technology, Volume 5, Pages 857-884, which text is accordingly incorporated herein by reference.

The edible films can also include one or more of the following agents: a triglyceride, a preservative, a polyethylene oxide compound, and propylene glycol. Examples of triglycerides include vegetable oils such as corn oil, sunflower oil, peanut oil, olive oil, canola oil, soybean oil and mixtures thereof. A preferred triglyceride is olive oil. The triglyceride is preferably added to the film in amounts from about 0.1 wt % to about 12 wt.%, preferably in a range from about 0.5 wt % to about 9 wt %, of the film. Preferred preservatives include sodium benzoate and potassium sorbate, preferably in amounts from about 0.001 wt % to about 5 wt %, preferably from about 0.01 wt % to about 1 wt % of the film. A preferred polyethylene oxide compound is N-10 having molecular weight from about 50,000 to about 6,000,000, which compound is available from Union Carbide Corporation. The polyethylene oxide compound is preferably added in amounts from about 0.1 wt % to about 5 wt %, preferably from about 0.2 wt % to about 4.0 wt % of the film. If propylene glycol is added, it is generally in amounts from about 1 wt % to about 20 wt %, preferably from about 5 wt % to about 15 wt % of the film.

The active ingredient used in the film can be coated to mask the taste of the active ingredient or to prevent the active ingredient from numbing the tongue or other surfaces in the oral cavity. The coatings that can be used are known to those skilled in the art. These include polymers such, as Eudragit™ acrylic polymers, cellulosics, such as ethylcellulose, and the like. An additional way to mask the taste of the active ingredient is by using an ion exchange resin such as Amberlite RP-69, available from Rohm and Haas, and Dow XYS-40010.00, available from the Dow Chemcial Co.

A film forming agent suitable for delivery of one gram of a sexual health or sexual dysfunction composition according to the invention can be formed from the following ingredients: 2.2 g pullulan, 8 mg xanthan gum, 10 mg locust bean gum, 42 mg carrageenan, 10 mg Sodium Benzoate, 70 mg acesulfame potassium, 500 mg lemon flavoring, 190 mg aspartame NF, 50 mg polysorbate 80 NF, 50 mg Atmos 300, 400 mg propylene glycol, 70 mg triglycerides, 35 mg titanium dioxide.

C. Description of the Preferred Food or Beverage Delivery System

The preferred sexual health and sexual dysfunction compositions according to the invention lend themselves to delivery in foods, beverages, powders, spices, condiments and salad dressings, particularly those that are intended for administration on a regular basis, such as a daily basis. It is difficult to take any supplement or medication on a daily basis. Many people have cupboards full of vitamins, supplements, and even medications that were purchased with the intent to take on a daily basis, but never were. Particularly since sexual encounters happen randomly and infrequently, the sexual health compositions of the invention may also sit in a cupboard, and when the time comes when they are needed, it may be too late. Thus, it is useful to have a vehicle and method of delivery of these compositions that is more natural and immediate, such as a food, beverage, spice condiment or salad dressing that is used every day.

Food, beverage, spices, condiments and salad dressing useful for delivery of nutraceutical compositions are described in U.S. patent application Ser. No. 10/616,674 filed Jul. 10, 2003 and U.S. patent application Ser. No. filed Jul. 10, 2004, which patent applications are hereby incorporated by reference to the same extent as though fully disclosed herein.

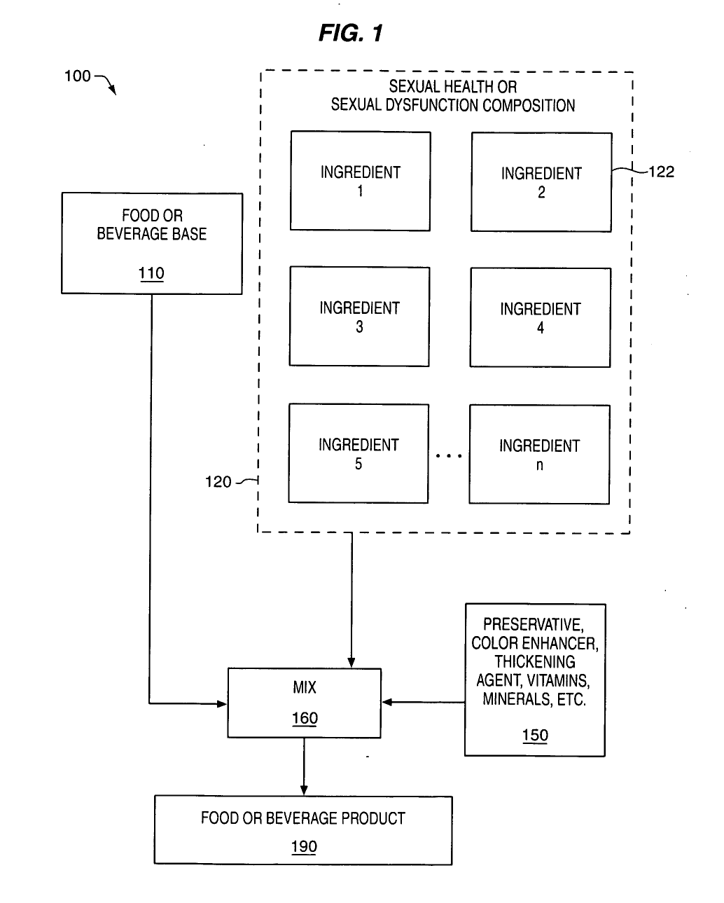

FIG. 1 illustrates the food or beverage product 190 according to the invention and the method of making it. The food or beverage product includes a food or beverage base 110 and a sexual health or sexual dysfunction composition 120, examples of which composition 120 are given above. Optionally, it may also include other ingredients 150, such as preservatives, color enhancers, thickening agents, vitamins, minerals, or other ingredients which may be added to foods. Food or beverage base 110, sexual health or sexual dysfunction composition 120, and any other desirable ingredient 150 are mixed at 160 to create food or beverage product 190.

The terms “food base” and “beverage base” as used herein are intended to include any and all normal foods and beverages as well as low-fat or light foods and beverages. For example, a low-fat or light salad dressing may be derived from the salad dressing base given below by substituting water and a thickening agent, such as xanthan gum, for all or a portion of the oil given in the formulation, or by other substitutions and formulations known in the salad dressing art. In this disclosure, all terms that relate to the food or beverage component have the meaning commonly used in the food art, and all terms that relate to the sexual health composition component have the meaning they commonly have in the nutraceutical art. Spices, condiments and salad dressings are intended to be included in the term “foods”, though we may at times specify these in particular to remind the reader that they are included in foods.

In the following, we will disclose a number of different food and beverage bases that have been found to be useful to make the food or beverage product 190. However, it should be understood that these are exemplary, that is, only illustrative, and not intended to be exhaustive.

In the above sexual health and sexual dysfunction compositions, the amounts of ingredients were given to make an appropriate dosage. In most cases, the amount given was the appropriate amount for a daily dosage. If it is expected that a food, foods, beverage, or beverages or both with the sexual health or sexual dysfunction composition added are expected to be consumed several times a day, the amounts used for a serving portion should be essentially halved—that is, it is assumed that the intake of the foods and/or beverages will be twice a day. It should be evident that if diets call for such foods or beverages more than twice a day, then the amounts of ingredients in a serving portion should be correspondingly reduced. If a food or beverage amount is such that it is intended for multiple servings, then the amount should also be correspondingly increased.

The food bases do not include chewing gum, inert ingredients by themselves, or other conventional materials for delivering nutraceuticals. The common definition of food necessarily includes only materials the provide essential body nutrients. That is, the definition of food is intended to be narrow and is not to be broadened to include materials not commonly recognized as food.

The preferred foods are: energy bars, salad dressings, condiments(such as steak sauce, mustard, catsup and soy sauce), vegetable oils, fruit products such as jellies, jams and syrups, cereals, trail mix, cookies, pasta, flours including wheat, soy, oat, and potato flour, whey, chocolate, yogurt, tofu, bagels, baked goods, vegetables, soups, trail mix, nutritional bars, snacks, crackers, meats, and meat products such as lunch meats, and milk products such as ice cream, cheese and butter.

Salad dressing provides a few detailed examples of the process and product of the invention. In these examples, when vegetable oil is mentioned, any vegetable oil may be used, preferably soybean oil or canola oil. In the following, “tsp” means teaspoon, “tbsp” means tablespoon, and “mg” is milligrams.

TABLE A
Oil And Vinegar Salad Dressing Base

½ cup water
½ cup vegetable oil
¼ cup vinegar (red wine vinegar may be used)
1 tsp sugar (optional)
½ tsp salt
½ tsp pepper

Small amounts of spices such as onion powder and/or garlic powder may be added.

TABLE B
Caesar Salad Dressing Base

1 egg, raw
3 tbsp lemon juice
garlic (about ½ teaspoon garlic powder or a large clove)
1 cup olive oil (other vegetable oil may be used)
¼ to ½ cup grated Parmesan or Romano cheese
1 tsp high fructose corn syrup
4 tbls anchovy paste
¼ tsp salt and pepper to taste

TABLE C
French Salad Dressing Base

½ cup vegetable oil
⅓ cup high fructose corn syrup
¼ cup water
3 tbls vinegar
½ tsp salt
½ tsp whey
⅓tsp modified food starch
pinch paprika
Enough yellow food color #5 and/or #6 to give desired color

The amounts given for exemplary salad dressings above are the amounts sufficient to fill a small bottle of about twelve to fifteen ounces. It is intended that the amounts of the ingredients given in the various salad dressing bases should be mixed with three to four dosages of the ingredients given in the various weight sexual health and sexual dysfunction compositions. The amounts are designed so that a serving of salad dressing is two ounces. It is assumed that salads are eaten twice a day so that two salads, or four ounces of salad dressing, provide effective daily amounts sexual health or sexual dysfunction ingredients. The various amounts given for salad dressing bases may be varied as known in the art of salad dressings, and the various amounts given for the sexual health and sexual dysfunction ingredients may be varied as known by those skilled in the art of sexual health and healthy, normal sexual function.

The above oil and vinegar salad dressing base, Caesar salad dressing base, and French salad dressing base are merely exemplary salad dressing bases. As is known in the art, there are many other variations of salad dressing bases, and particularly many other variations of oil and vinegar salad dressing bases, Caesar salad dressing bases, French salad dressing bases, Ranch salad dressing bases, Bleu Cheese salad dressing bases, Russian salad dressing bases, and Thousand Island salad dressing bases, all of which may be used in the invention. From the above disclosure, and the sexual health and sexual dysfunction compositions given above, those skilled in the art of foods will understand the invention and how to implement it in a wide variety of foods.

The term “beverage” is used in its common meaning which does not include water or medicines. The preferred beverages are sports drinks, soft drinks, alcoholic beverages, tea, coffee, milk, and fruit juices.

A well-known formula for a basic sports drink is: 1 quart (32 oz) or 1 liter water, ⅓ cup sugar, ¼ teaspoon sodium chloride, flavoring to taste. Potatssium chloride may be substituted for some of the sodium chloride if potassium levels are a concern. Any of the flavorings mentioned in section 3B above may be used.

Another general purpose sports beverage that is particularly useful for athletic persons that exercise heavily for long periods, can be made using the following ingredients: saccharose 55 g/l, oligosaccharides 20 g/l, citric acid 1.8 g/l, citric aroma 1.0, sodium chloride 1.0 g/l. The foregoing components can be mixed in the dry state and dissolved in 1 liter of water.

In the preferred embodiment, the dry ingredients for the beverage are mixed with two doses of the desired sexual health composition and the mixed combination is dissolved in one liter or one quart of water.

From the above disclosure, and the sexual health and sexual dysfunction compositions given above, those skilled in the art of beverages will understand the invention and how to implement it in a wide variety of beverages.

Any of the above-mentioned foods or beverages, as well as other foods or beverages, can be used as a base for the preparation of a functional food or functional beverage product according to the invention. A sexual health or sexual dysfunction composition as disclosed above is mixed with the food or beverage base in appropriate amounts to provide the approximate dosages given above in a serving, or a plurality of servings if the food or beverage is expected to be ingested more often.

The method of the invention preferably involves the administration, preferably daily oral administration, of the above-described compositions in conjunction with a specific dietary plan in a synergistic format. The dietary plan preferably involves multiple small meals, each reflecting the overall macronutrient composition of the diet. Carbohydrate content is low as is the glycemic index. Protein content is high. In the dietary regimen followed in the present invention, the glycemic index is preferably less than 60, more preferably less than 45; the carbohydrate content will constitute less than 50%, more preferably 0%-50%, most preferably about 7%-40% of the calories consumed on a daily basis; and the protein intake constitutes preferably at least 20%, more preferably about 20% -40%, most preferably about 25-35% of total daily caloric intake. The number of meals is preferably 2, more preferably 4 to 6, per day. Adequate fluid intake is recommended to insure excellent hydration.

As stated above, the nutraceutical compositions of this invention are preferably administered on a daily basis. However, if desired, the composition can be administered on a non-daily basis, e.g., on alternate days . The frequency of administration will depend on the sexual needs of the user. The more frequent the composition is administered, the faster the attainment of healthy sexual function. Thus, daily administration of the composition will result in faster achievement of sexual health than non-daily administration.

The method of this invention preferably further includes an exercise program. Preferably, the exercise program will be followed at least 2 days a week and more preferably three to five days per week. The exercise program should preferably include components of aerobic and resistance training as tolerated by the individual in need thereof. The exercise program increases the over-all well being of the user which aids sexual performance, which leads to sexual confidence.

The invention may further include a stress reduction program designed to diminish glucocorticoid activity. By downgrading glucocorticoid activity, the stress reduction program acts to improve enjoyment of sex with concurrent improvements in sexual function. Utilizing similar mechanisms, pyridoxine augments both these actions. The stress reduction program may involve any activity that lowers glucocorticoid levels. Non-limiting examples of such activities include relaxation, getting a massage, acupuncture, psychotherapy, meditation, taking a sedative, and the like.

There have been described novel compositions and methods for enhancing sexual health. It should be understood that the specific formulations and methods described herein are exemplary and should not be construed to limit the invention, which will be described in the claims below. Further, it is evident that those skilled in the art may now make numerous uses and modifications of the specific embodiments described without departing from the inventive concepts. For example, any of the sexual performance or sexual dysfunction pharmaceuticals, such as vardenafil, tadalafil, sildenafil, and apomorphine, can also be administered by one of the vehicles disclosed in section 3 above. Further, only exemplary additives, such a breath-freshening agents, have been mentioned, since this disclosure is not about breath-freshening, but compositions and methods for healthy sexual function. To include all possible additives, such as all possible breath-freshening agents, would make the disclosure unwieldy and detract from the essential focus. Consequently, the invention is to be construed as embracing each and every novel feature and novel combination of features present in and/or possessed by the compositions and methods described and by their equivalents.

1. A product for supporting and promoting healthy sexual function, for preventing human sexual dysfunction, for preventing human sexual dysfunction, or for treatment of human sexual dysfunction, said product comprising:
a delivery vehicle selected from the group consisting of edible films, breath-care strips, mints, and breath-care lozenges; and
a sexual health or sexual dysfunction composition carried by said vehicle in an effective amount for supporting and promoting healthy sexual function, for preventing human sexual dysfunction, or for treatment of human sexual dysfunction.

2. A product as in claim 1 wherein said sexual health or sexual dysfunction composition comprises an ingredient selected from the group consisting of: a portion of the plant Sophora flavescens or an extract of the plant Sophora flavescens, said ingredient provided in an effective amount for supporting and promoting healthy sexual function, for preventing human sexual dysfunction, or for treatment of human sexual dysfunction.

3. A product as in claim 1 wherein said sexual health or sexual dysfunction composition comprises sophoflavescenol in an effective amount for supporting and promoting healthy sexual function, for preventing human sexual dysfunction, or for treatment of human sexual dysfunction.

4. A composition as in claim 3 wherein said composition further comprises an ingredient that enhances the amount, or activity, of guanylate cyclase, an ingredient that enhances the production of nitric oxide, or an ingredient that inhibits the breakdown of nitric oxide; said ingredients included in effective amounts for supporting and promoting healthy sexual function, for preventing human sexual dysfunction, or for treatment of human sexual dysfunction.

5. A composition as in claim 4 wherein said ingredient that enhances the amount, or activity, of guanylate cyclase comprises biotin, said ingredient that enhances the production of nitric oxide comprises a compound selected from the group consisting of L-arginine and policosanol, and said ingredient that inhibits the breakdown of nitric oxide comprises an anti-oxidant selected from the group consisting of idebenone, co-enzyme Q10, vitamin E, ALA, vitamin C, carnosine, tocotrienols, flavonoids, ALC, vinpocetine, selenium, lycopene, creatine, arginine, taurine, cysteine, NADH, resveratrol, ginkgo biloba, oligomeric proanthocyanidins, and phenolic antioxidants and folic acid, folate, folinic acid and tetrahydrofolate.

6. A product as in claim 1 wherein said sexual health or sexual dysfunction composition comprises a Vitex agnus-castus extract in an effective amount for supporting and promoting healthy sexual function, for preventing human sexual dysfunction, or for treatment of human sexual dysfunction.

7. A product as in claim 1 wherein said sexual health or sexual dysfunction composition comprises a cGMP-specific PDE5 inhibitor.

8. A product as in claim 7 wherein said cGMP-specific PDE5 inhibitor comprises an ingredient selected from the group consisting of, vardenafil, tadalafil, and sildenafil.

9. A product as in claim 1 wherein said sexual health or sexual dysfunction composition comprises a dopaminergic agonist.

10. A product as in claim 11 wherein said dopaminergic agonist comprises apomorphine.

11. A product for supporting and promoting healthy sexual function, for preventing human sexual dysfunction, for preventing human sexual dysfunction, or for treatment of human sexual dysfunction, said product comprising an edible film containing a sexual health or sexual dysfunction composition in an effective amount for supporting and promoting healthy sexual function, for preventing human sexual dysfunction, or for treatment of human sexual dysfunction.

12. A product as in claim 11 wherein said sexual health or sexual dysfunction composition comprises a cGMP-specific PDE5 inhibitor.

13. A product as in claim 12 wherein said cGMP-specific PDE5 inhibitor comprises an ingredient selected from the group consisting of sophoflavescenol, vardenafil, tadalafil, and sildenafil.

14. A product as in claim 11 wherein said sexual health or sexual dysfunction composition comprises Vitex agnus-castus extract.

15. A product as in claim 11 wherein said edible film comprises pullulan.

16. A food or beverage product for supporting and promoting healthy sexual function, for preventing human sexual dysfunction, for preventing human sexual dysfunction, or for treatment of human sexual dysfunction, said product comprising:
a food or beverage base; and
a sexual health or sexual dysfunction composition in an effective amount for supporting and promoting healthy sexual function, for preventing human sexual dysfunction, or for treatment of human sexual dysfunction, said composition selected from the group consisting of: a portion of the plant Sophora flavescens, an extract of the plant Sophora flavescens, sophoflavescenol, vardenafil, tadalafil, sildenafil, apomorphine, and Vitex agnus-castus extract.

17. A product as in claim 16 wherein said base is a beverage base and said beverage is selected from the group consisting of: soft drinks, tea, coffee, milk, fruit juices, and sports drinks.

18. A product as in claim 16 wherein said base is a beverage base and said beverage comprises an alcoholic beverage.

19. A product as in claim 16 wherein said base is a food base and said food is selected from the group consisting of: fruit, chocolate, candy, trail mix, nutritional bars, ice cream, yogurt, cheese, steak sauce, salsa and salad dressing.

20. A method of administering a composition for supporting and promoting healthy sexual function, for preventing human sexual dysfunction, for preventing human sexual dysfunction, or for treatment of human sexual dysfunction, said method comprising:
incorporating said composition in an edible film, breath-care strip, mint, or breath-care lozenge; and
placing said edible film, breath-care strip, mint, or breath-care lozenge in the mouth of a human being.
